(19) United States
(12) Patent Application Publication
(10) Pub. No.: US 2006/0127458 A1
(43) Pub. Date: Jun. 15, 2006

(54) Single use personal care sheet

(76) Inventors: Melba Kiser, Flemington, NJ (US); Robert Bianchini, Hillsborough, NJ (US); Richard Tokosh, Saddle Brook, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/012,445

(22) Filed: Dec. 15, 2004

(51) Int. Cl. A61K 9/70

(52) U.S. Cl. 424/443

(57) ABSTRACT

The invention relates to a single use cleansing sheet comprising: (a) a first fibrous water soluble polymer; (b) a second fibrous water soluble polymer which is different from said first fibrous water soluble polymer; wherein the ratio of (a) and (b) is effective to provide a cleansing sheet which wets out almost immediately upon contact with water; (c) a plasticizer in an amount effective to increase the flexibility and strength of the sheet; (d) a surfactant portion present in an amount effective to provide cleansing and lathering properties; and (e) water. In another embodiment, the invention relates to a method for cleansing the hands, face, hair, or body comprising wetting the cleansing sheet according to the invention with water, applying mild friction and contacting hands, face or body with the wetted sheet. In yet another embodiment, the invention relates to a single use personal care sheet comprising: (a) a first fibrous water soluble polymer; (b) a second fibrous water soluble polymer which is different from said first fibrous water soluble polymer (a); wherein the ratio of (a) and (b) is effective to provide a personal care sheet which wets out almost immediately upon contact with water; (c) a plasticizer in an amount effective to increase the flexibility and strength of the sheet; (d) a combination of at least one hydrophobic and at least on hydrophilic emulsifier in an amount effective to form a stable emulsion; and (e) water. In another embodiment, the invention relates to a method for treating,. i.e., moisturizing, protecting, soothing, etc., the hands, face, hair, or body comprising wetting the personal care sheet according to the invention with water, applying mild friction and contacting hands, face or body with the wetted sheet.

### I. FIELD OF THE INVENTION

The invention relates to a personal care sheet, e.g., cleansing sheet, having superior lathering properties, suitable for use as a facial cleanser and sufficiently pliable so that it will not break on transit, or during removal from the package, can be obtained by using a fibrous water soluble polymer portion comprising methylcellulose and at least one other fibrous water soluble polymer.

### II. BACKGROUND OF THE INVENTION

Due to sanitary concerns the demand for paper soaps has increased because it provides no concerns for cross contamination because the sheet is disposable and therefore can be limited to use by one person. Such “paper soaps” have been marketed for many years. For example, U.S. Pat. No. 2,356,168 discloses soap leaves or sheets consisting of methyl celluose and soap. However, the paper soap products are affected by humidity which causes tackiness and stickiness which lead the products to stick together and may be too brittle to be practically used—limited shelf life. Further, such paper soap products are harsh to the skin due to the alkalinity of soap bases. Additionally, due to the skin irritation associated with soap based products, paper soaps have limited use and the current products are not suitable for facial use. Therefore, it has been desired to develop a product that is suitable for facial use, is mild to the skin, has good foaming properties and has fast solubility during product use.

### III. SUMMARY OF THE INVENTION

It has been discovered that a personal care sheet, e.g., cleansing sheet, having superior lathering properties, suitable for use as a facial cleanser and sufficiently pliable so that it will not break on transit, or during removal from the package, can be obtained by using a fibrous water soluble polymer portion comprising methylcellulose and at least one other fibrous water soluble polymer.

Accordingly, the invention relates to a single use cleansing sheet comprising:
(a) a first fibrous water soluble polymer;
(b) a second fibrous water soluble polymer which is different from said first fibrous water soluble polymer (a); wherein the ratio of (a) and (b) is effective to provide a cleansing sheet which wets out almost immediately upon contact with water;
(c) a plasticizer in an amount effective to increase the flexibility and strength of the sheet;
(d) a surfactant portion present in an amount effective to provide cleansing and lathering properties; and
(e) water.

In another embodiment, the invention relates to a method for cleansing the hands, face, hair, or body comprising wetting the cleansing sheet according to the invention with water, applying mild friction and contacting hands, face or body with the wetted sheet.

In yet another embodiment, the invention relates to a single use personal care sheet comprising:
(a) a first fibrous water soluble polymer;
(b) a second fibrous water soluble polymer which is different from said first fibrous water soluble polymer (a); wherein the ratio of (a) and (b) is effective to provide a personal care sheet which wets out almost immediately upon contact with water;
(c) a plasticizer in an amount effective to increase the flexibility and strength of the sheet;
(d) a combination of at least one hydrophobic and at least one hydrophilic emulsifier in an amount effective to form a stable emulsion; and
(e) water.

In another embodiment, the invention relates to a method for treating, i.e., moisturizing, protecting, soothing, etc., the hands, face, hair, or body comprising wetting the personal care sheet according to the invention with water, applying mild friction and contacting hands, face or body with the wetted sheet.

### IV. DETAILED DESCRIPTION OF THE INVENTION

A. The Fibrous Water Soluble Polymer

As discussed above, it has been discovered that a cleansing sheet having superior lathering properties, suitable for use as a facial cleanser and sufficiently pliable so that it will not break on transit or during removal from the package can be obtained by using (a) a first fibrous water soluble polymer; (b) a second fibrous water soluble polymer which is different from said first fibrous water soluble polymer (a); wherein the ratio of (a) and (b) is effective to provide a cleansing sheet which wets out almost immediately upon contact with water and mild friction; (c) a plasticizer in an amount effective to increase the flexibility and strength of the sheet; (d) a surfactant portion present in an amount effective to provide cleansing and lathering properties; and (e) water.

By “wets out” is meant the cleansing sheet disintegrates into minute particles. By “mild friction” means rubbing between the hands and/or fingers or rubbing between the hands or fingers and the object to be cleaned, e.g., face. By “almost immediately” means less than one minute, for example, less than 30 seconds, less than 15 seconds, and even less than 5 seconds after contact with water.

Suitable fibrous water soluble polymers used in the cleansing and/or personal care sheet of the invention include methylcellulose, available for example, from DOW Chemical Co. under the name METHOCEL K35, hydroxypropyl cellulose, such as, METHOCEL A15LV available from DOW Chemical Co., ethyl 2-hydroxyethyl ether cellulose available from AKZO NOBEL, carboxycellulose, polyvinyl alcohol, hydroxypropyl methyl cellulose available from DOW Chemical Co. under the name METHOCEL Premium E5 LV and K35 LV, carboxymethyl cellulose, polyvinylpyrrolidone and polyacrylates (e.g. sodium, potassium or ammonium salts) and the like. In one embodiment, the single use cleansing and/or personal care sheet according to the invention is free of hydroxy ethyl cellulose.

It has been discovered that when the fibrous water soluble polymer consists entirely of methyl cellulose the resulting sheet curls after drying and is therefore unacceptable. Furthermore, it has been discovered that when the fibrous water soluble polymer consists entirely of hydroxypropyl cellulose the resulting sheet was also unacceptable because the product ripped and was mottled in appearance. Such a product would likely crack after packaging and would have a high potential of breaking when removed from the package. Accordingly, one embodiment of the invention, the fibrous water soluble polymer is a mixture of (a) methyl cellulose and (b) at least one other fibrous water soluble polymer. The ratio of methylcellulose to the at least one other fibrous water soluble polymer should be effective to provide a cleansing sheet which almost immediately wets out upon contact with water and mild friction so lather generation can be easily achieved. In another embodiment, the ratio of methylcellulose to said at least one other water soluble polymer ranges from about 4:1 to less than about 1:4, for example, from about 4:1 to about 1:1. In another embodiment, the at least one other fibrous water soluble polymer is hydroxypropyl cellulose.

Generally, the amount of fibrous water soluble polymer ingredient ranges from about 5% to about 30%, for example, 5% to 20%, for example, 8% to about 12% preferably 10% by weight wet basis (i.e., before drying), based on the total composition.

The ratio of water to water soluble fibrous polymer ingredient in the cleansing sheet according to the invention ranges from about 95:5 to about 50:50, for example from about 80:20 to about 85:15.

B. The Plasticizer

The cleaning sheets according to the invention comprise a plasticizier which increases the flexibility and strength of the cleansing sheet. In some cases, the plasticizer also provides skin conditioning benefits and added emolliency. Any ingredient that is capable of increasing the flexibility and strength of a cleansing sheet may be incorporated as a plasticizer into the cleansing sheets according to the invention so long as it does not have a detrimental effect on the skin and is readily dispersed within the formulation prior to drying.

Examples of suitable plasticizers include glycerine, glycols such as ethylene glycol, propylene glycol, butylene glycol and diethylene glycol and polyglycols such as polyethylene glycols and polypropylene glycols (e.g., PPG-425), starches, sugars, dextrin, sorbitol, glucose amine, and salts of ethanolamines such as ethanolamine stearate. Generally, the plasticizer is present in an amount effective to provide a cleansing sheet with sufficient flexibility and strength depending upon its intended use. In one embodiment, the plasticizer ranges from about 0.1 to about 20% by weight, for example, from about 0.25 to about 10%, preferably from about 4 to about 8% wet basis (i.e., before drying) based on the total composition.

C. The Surfactant Portion

The cleansing sheet according to the invention further comprises a surfactant portion to provide cleansing and lathering benefits. The surfactant portion may be a single surfactant or a mixture of surfactants and may be selected from anionic, cationic, amphoteric and nonionic surfactants. The surfactant or mixture of surfactants should not have a detrimental effect on the skin and should be readily dispersed in the formulation prior to drying.

Generally, the surfactant portion is present in an amount effective to provide cleansing and lathering properties to the cleansing sheet. In one embodiment of the invention, the surfactant portion is present at from about 20% to about 80%, for example from about 30% to about 75%, and from about 40% to about 70% by weight on a dry basis (i.e., dried sheet), based on the total composition.

Due to the skin irritation associated with soap based products, in a preferred embodiment, the cleansing sheet according to the invention is substantially free of natural soaps. By substantially free is meant less than about 10% by weight, for example less than about 5% by weight, on a dry basis, based on the totally composition. In one embodiment, the cleansing sheet is free of natural soaps.

The ratio of surfactant portion to fiber forming water soluble polymer portion ranges from about 50:50 to about 90:10, for example, from about 70:30 to about 80:20.

Surfactants suitable for use in the cleansing sheet according to the invention include anionic, cationic, amphoteric and nonionic surfactants. Examples include alkyl sulfates, ethoxylated alkyl sulfates, betaines, taurates, sarcosinates, sulfosuccinates, alkyl sulfates, and monoalkylphosphates. Specific examples include but are not limited to sodium laureth sulfate, cocamidopropyl betaine, sodium methyl cocoyl taurate, sodium cocoyl sarcosinate, disodium laureth sulfosuccinate, magnesium laureth sulfate, sodium laureth-8 sulfate, magnesium laureth 8 sulfate, sodium oleth sulfate and magnesium oleth sulfate, and mixtures thereof.

In one embodiment of the invention, the cleansing sheet is particularly suited for use as a personal cleanser, i.e., for cleansing the hands, face and body. In this embodiment, the surfactant portion of the cleansing sheet according to the invention should contain surfactant(s) that provide cleansing and lathering properties but are also mild to the skin.

The surfactant portion may also comprise a high foaming surfactant. Generally, the high foaming surfactant is present in a weight ratio of high foaming surfactant to fibrous water soluble polymer portion of from about 50:50 to about 90:10, for example, from about 70:30 to about 75:25. An example of a particularly suitable high foaming surfactant includes sodium laureth ether sulfate. Other surfactants such as disodium lauroamphodiacetate available from Uniqema under the name MONATERIC 949J, betaines such as cocoamidopropyl betaine and monoalkyl phosphates may also be used.

The surfactant portion may also comprise an irritation reducing surfactant to improve the mildness of the cleansing sheet. The weight ratio of the irritation reducing surfactant to fibrous water soluble polymer portion ranges from about 10:90 to about 80:20, for example, from about 35:65 to about 40:60. An example of a particularly suitable irritation reducing surfactant includes cocamidopropyl betaine, sodium methyl cocoyl taurate, disodium lauramphodiacetate and monoalkylphosphates. Sodium methyl cocoyl taurate is not only a suitable irritation reducing surfactant but also beneficially improves the lathering characteristics and provides better skin after feel after product use. An example of an acceptable range of the irritation reducing surfactant is from about 1 to about 15% by weight, for example, from about 5 to about 15% by weight.

The surfactant portion may also comprise a mild foam booster, e.g., cocamidopropyl betaine and sodium methyl cocoyl taurate and mono-alkylphosphates

Another ingredient that is beneficial to the invention is a wetting agent to improve the solubility of the cleansing sheet. Examples include disodium laureth sulfosuccinate (manufactured by Huntsman Chemical Corp. under the trade name of Chemccinate DSLS and Ultrasurf DSLS available from Ultra Chemical). The weight ratio of the wetting agent to fibrous ingredients preferably ranges from about 10/90 to 50/50 with the ideal range being 15/85 to 25/75.

In one embodiment of the invention, the surfactant portion comprises:
a. sodium laureth sulfate, present in a weight ratio of sodium laureth sulfate to fibrous water soluble polymer portion of from about 50:50 to about 90:10, for example, from about 70:30 to 75:25;
b. cocamidopropyl betaine present in a weight ratio of cocoamidopropyl betaine to fibrous water soluble polymer portion of from about 20:80 to about 80:20, for example, from about 35:65 to about 40:60;
c. sodium methyl cocoyl taurate, present in a weight ratio of sodium methyl cocoyl taurate to fibrous water soluble polymer portion of from about 10:90 to about 80:20, for example, from about 25:75 to about 35:65
d. sodium cocoyl sarcosinate present in a weight ratio of sodium cocoyl sarcosinate to fibrous water soluble polymer portion of from about 10:90 to about 80:20, for example, from about 16:84 to about 18:82.
e. disodium laureth sulfosuccinate present in a weight ratio of disodium laureth sulfosuccinate to fibrous water soluble polymer portion of from about 10:90 to about 50:50, for example from about 20:80 to about 25:75.

D. Optional Beneficial Ingredients

The cleansing sheet according to the invention may optionally comprise additional ingredients which enhance the properties of the cleansing sheet and provide useful benefits depending upon the intended use of the cleansing sheet. Such ingredients, when utilized, should be present in an amount effective to provide the desired benefit.

An example of an ingredient that is beneficial to the invention and may be added is a viscosity adjuster which is present in amount effective to achieve the desired viscosity adjustment. Any ingredient that is capable of changing the viscosity of the composition may be incorporated. For example, it has been discovered that contrary to the vendor's literature, Sulfochem ASV-K manufactured by Chemron Corp., a proprietary blend of sodium laureth sulfate, magnesium laureth sulfate, sodium laureth-8 sulfate, magnesium laureth-8 sulfate, sodium oleth sulfate and magnesium oleth sulfate can be used to decrease the viscosity of the formulation. Although the manufactures literature states that Sulfochem has “viscosity building” properties, it was surprisingly discovered that when added to the batch it actually decreased the viscosity. This reduction in viscosity significantly improved the processing characteristics of the cleansing sheet. The percentage of Sulfochem ASV-K in the formula ranges from about 0.01% to about 5.0%, for example, from about 0.1% to about 0.2% wet basis.

When the cleansing sheet according to the invention is intended for use as a personal cleanser, skin benefit agents may be particularly useful. Examples of skin benefit agents include exfoliating and/or scrubbing ingredients (e.g., beads, pumice, talc, silica). Examples of suitable exfoliating/scrubbing ingredients include but are not limited to Inert, hydrophobic, high-density polyethylene beads having a particle size ranging from about 20 to about 500 microns, for example from about 150 to about 315 microns. An example of which is INDUCOS 14/2 available from Induchem AG, Volketswil, Switzerland.

Also suitable as skin benefit agents are skin protectants ( e.g., Snow White Petrolatum, dimethicone, calamine), diaper rash treatment (e.g., Zinc Oxide, USP), occlusives (e.g., Cocoa Butter, hydrogenated oils, propylene glycols), absorbents (e.g., bentonite, hectorite, potato starch, calcium silicate, diatomaceous earth, kaolin), biological additives from the class of vegetable, marine and fruit extracts, such as, for example, algae extract, arnica extract, carrot seed extract, eucalyptus extract, peppermint extract, grape extract, mugwort extract, oak bark extract, acne treatment agents (e.g., Salicylic Acid, USP), skin soothing ingredients (e.g., polymeric emulsifiers oatmeal, panthenol, allantoin, aloe, etc.), conditioning agents (e.g., avocado oil, isopropyl myristate, jojoba esters, mineral oil, squalene) sun screen agents, soy and perfumes. Examples of suitable polymeric emulsifiers useful as skin soothing ingredients include but are not limited to copolymers of acrylic acid modified by long chain (C₁₀-C₃₀) alkyl acrylates and crosslinked with allylpentaerythritol sold under the name Pemulen TR-1 NF by Noveon, Inc. Cleveland, Ohio. Another suitable skin soothing agent is sodium astrocaryum murumuruate (saponified triglycerides of Astrocaryum sp palm trees) available under the name CHEMYSOAP by Chemyunion Quimica LTD.

When salicylic acid is incorporated into the cleansing sheet according to the invention, it is preferably added to water prior to the addition of fibrous water soluble polymer and the water is heated to a temperature sufficient to solubilize the salicylic acid, for example, at about 90° C.

In one embodiment, the weight ratio of skin soothing agent to the fibrous water soluble polymer portion ranges, for example from about 20:80 to 80:20 with the ideal range being from about 25:75 to about 30:70.

As used herein the term “perfume” includes any non-water soluble fragrant substance or mixtures of ingredients that are natural (e.g., extracts of flowers, herb or plants), artificial (e.g., natural oils or oil constituents) and synthetically produced substances that emit an odor. Typically, perfumes are complex mixtures of organic compounds such as alcohols, aldehydes, ethers, aromatic compounds and essential oils such as terpenes. In the present invention the precise composition of the perfume is not critical to the product performance as long as it meets the criteria of water disperseability and has a pleasant odor. In place of the perfume an essential oil can be used or a water insoluble hydrocarbon.

The cleansing sheet according to the invention may also contain other compounds to provide additional effect or to make the product more attractive to the customers. The following are examples: colors or dyes in the amount up to 1.0% by weight, antibacterial agents such as Irgasan DP300 or TCC ranging in concentration from 0.1% to 2.0%, extracts such as Aloe from 0.1% to 2.0%, pH adjusting agents such as citric acid, lactic acid and sodium hydroxide.

In one embodiment of the invention, the cleansing sheet according to the invention, has a pH ranging from about 3 to about 8, for example, from about 5.5 to about 6, wherein the pH is measured on the “wet batch” before the drying process with a standard pH meter. When adding salicylic acid the pH ranges, for example, from about 3.5 to about 4.5.

E. Methods of Use

The cleansing sheet according to the invention may be useful to clean a variety of surfaces, for example, as hard surface cleansers and personal cleansers. In using the detergent sheet of the present invention, the cleansing sheet is wetted and then mild friction is applied, i.e., rubbed between the hands and/or fingers or between the hands and/or fingers and the surface to be cleaned. The mild friction not only produces lather but also releases any additional benefit agents that may be present within the sheets. It also causes the fibrous water soluble polymer portion to wet out almost immediately upon contact with water.

In one embodiment, the invention relates to a method for cleansing the hands, face, hair, or body comprising wetting the cleansing sheet according to the invention with water, applying mild friction and contacting hands, face or body with the wetted sheet.

Because the cleansing sheets according to the invention are mild to the skin but also provide superior lather, they are particularly suited for use as a facial cleanser and/or as a baby product, such as, for example, a shampoo or a skin cleanser for the diaper area.

In yet another embodiment of the invention, the sheets according to the invention may be formulated as a single use personal care sheet useful as a moisturizing lotion, sun care lotion, diaper rash lotion and/or any consumer product that would provide benefits if formulated into a single use sheet that could be wetted with water and then applied to the area to be treated. Accordingly, in another embodiment, the invention relates to a single use personal care sheet comprising:
(a) a first fibrous water soluble polymer;
(b) a second fibrous water soluble polymer which is different from said first fibrous water soluble polymer (a); wherein the ratio of (a) and (b) is effective to provide a cleansing sheet which wets out almost immediately upon contact with water;
(c) a plasticizer in an amount effective to increase the flexibility and strength of the sheet;
(d) a combination of at least one hydrophobic and at least one hydrophilic emulsifier in an amount effective to form a stable emulsion;
(e) water.

Suitable emulsifiers include naturally occuring fatty acid such as Chemysoap, naturally occurring waxes such as bees wax, irritation reducing surfactants, such as dimethicone copolyol, Dow Corning 3225C, as well as the skin protectants, skin soothers, and irritation reducing surfactants described above.

F. Methods of Making

The cleansing sheet according to the invention may be made by blending the surfactant portion, the fibrous water soluble polymer portion, the plasticizer and water. This mixture is applied to a substrate, e.g., a non-water soluble transfer paper that does not stick to the finished product after drying. After the mixture is applied to the non water soluble transfer paper, the product moisture is reduced by passing the product through hot air ovens. Generally, the amount of water in the dried sheet ranges from about 0.5 to about 7% by weight, for example from about 1 to 5%, for example from about 3 to 4% by weight. After drying, the product is released from the paper, cut into desired lengths and shapes.

The following are four different examples of suitable processes useful for making the cleansing sheets according to the invention:

Process A:

In a main vessel, add water, plasticizer and surfactant and start mixing. Blend the two fibrous water soluble polymers together and add to the water, plasticizer and surfactant mixture with mixing. Continue mixing to ensure all ingredients are homogenous within the mixture. The mixture is then heated to about 50 to about 60° C. Heat is discontinued and mixing continues while the remaining ingredients are added allowing the mixture to cool with each addition. Any perfume and additional optional benefit agents are added last.

Process B:

In this process all surfactants are added to the water phase followed by the plasticizer, preservatives if any and fragrance with mixing. Once the mixture is homogeneous the fibrous water soluble polymers are added. Mixing continues until all ingredients are well dispersed and there are no agglomerates present. Because no heating is required, this process is an energy efficient process with less air incorporated in the batch.

Process C:

In this process water is heated to about 70 to about 90° C., for example about 80° C., the fibrous water soluble polymers are added and mixed until totally homogeneous (lump free). Once the mixture is homogeneous all of the surfactants, plasticizer, preservatives, fragrance and additional ingredients are added and mixed until homogeneous.

Experimentation demonstrated that temperature during the preparation of the batch has a dramatic effect on the thickness and uniformity of the feedstock. When the batch was prepared at about 50° C., or lower, i.e., as in Process A and Process B) the product was thick and not totally uniform. The consistency made it difficult to apply the base stock onto the paper for the drying operation.

When the batch was made hot at about 80° C., i.e., as in Process C, the feedstock was totally homogeneous and thinner (it was pumpable at about 50° C. and it was easy to pour). For these reasons, the application to the paper was superior to Process A and B that, in turn, produces a better looking product.

Process D:

In this process water is heated to approximately 90° C. If the formula requires salicylic acid, it is added to the hot water and mixed until dissolved. The fibrous water soluble polymers are added to the hot water and mixed for 5 minutes to 45 minutes, preferably 20 minutes, to ensure the fibrous water soluble polymers are wetted and totally dispersed thought-out the batch. After the fibrous water soluble polymers are wetted at high temperature the temperature is reduced to approximately 5° C. to 50° C., preferably about 20±5° C. The mixture is held at this temperature for approximately 10 minutes to 90 minutes, preferably 40 minutes. The plasticizing agents are then added followed by the surfactants. These ingredients can be added individually, or mixed together until homogeneous, and then added as a blend to the batch. When all of the ingredients are added to the batch the composite is mixed for 5 minutes to 120 minutes, but more preferably for 20 minutes, or until homogeneous. The fragrance and any additional benefit agent is added last. After the batch is completed it is milled in a homogenizer until the specific gravity of the composite is 0.2 grams/cc to 0.9 grams/cc but more preferably at 0.7±0.1 grams/cc.

It has been discovered that incorporating “air” into the formulation will dramatically improve the solubility of the cleansing sheet according to the invention. Specifically, it has been discovered that product solubility can be dramatically improved by reducing the “specific gravity” of the formula by homogenizing the batch, using for example a high shear mixer such as a Gifford-Wood homogenizer available from J.W. Greer Inc., Hudson, N.H. In one embodiment of the invention, the specific gravity of the cleansing sheet according to the invention ranges from about 0.4 to about 0.97, for example, from about 0.6 to about 0.9, for example, about 0.7, as measured with a Fischer Scientific Pycnometer.

Several examples are set forth below to further illustrate the nature of the invention and the manner of carrying it out. However, the invention should not be considered as being limited to the details thereof.

### V. EXAMPLES

The cleansing and personal care sheets described in the following examples were prepared using the following general process:

Process for the Examples:

1. Water was added to a main tank and turbine mixing was started at low speed and then heated to 70° C. (when salicylic acid is incorporated it was added to water prior to the addition of fibrous water soluble polymer and the water was heated to a temperature sufficient to solubilize the salicylic acid, for example, at about 90° C.).
2. While heating glycerin was added to the water and mixing continued. Sulfochem was added to the water and glycerin. Mixing continued with heat until the temperature of the water phase was at 70° C.
3. Half of the sodium lauryl sulfate (“SLS”) was added to the batch to help solubilize the fibrous water soluble polymers.
4. With the water phase at 70° C., the two fibrous water soluble polymers were added to the water phase. Mixing was increased if necessary in order to incorporate the powders into the water phase and then allowed to mix for 15 to 20 minutes.
5. The heat was turned off and and cooling started gradually. The following chemicals were then added to help cool the batch:
   (d) Second part of the SLS
   (e) Tegobetaine
   (f) Hampocyl
   (g) Tauranol
   (h) Chemcinate DSLS
   (i) Phenonip
6. When the batch temperature was about 70° C.±2° C., Euperlan and fragrance were blended in along with any additional benefit agent.
7. Mixing continued for at least 15 minutes or until batch was homogeneous, whichever was more.
8. The mixture was applied to a non-water soluble transfer paper and dried.

### Example I

### Cleansing Sheet

A single use cleansing sheet was prepared by the above process and containing the ingredients set forth in the following table:

| | | | Wt. % | Wt. % |
|---|---|---|---|---|
| | | Amount | (Wet | (Dried |
| | | (grams) | Formula) | product) |
| Water | Water | 45.900 | 42.817 | 3.361 |
| Methylcellulose | Methocel | 8.000 | 7.463 | 25.085 |
| | A125LV | | | |
| Blend of | Sulfochem | 0.200 | 0.187 | 0.314 |
| Sulfates | ASV-K | | | |
| | (50% active) | | | |
| Hydroxypropylcellulose | Methocel | 2.000 | 1.866 | 6.272 |
| | K35 LV | | | |
| Sodium Laureth | Rhodapex | 27.000 | 25.187 | 22.011 |
| Sulfate | ES-2K | | | |
| | (26% active) | | | |
| Cocamidopropyl | Tegobetaine | 6.000 | 5.597 | 5.644 |
| betaine | L7-V | | | |
| | (30% active) | | | |
| Glycerin, USP | Emery 917 | 4.000 | 3.731 | 12.541 |
| Phenoxyethanol | Phenonip | 0.300 | 0.280 | 0.941 |
| & parabens | | | | |
| Fragrance | IFF 303 AV | 0.800 | 0.746 | 2.507 |
| | PCM RPD | | | |
| Sodium methyl | Tauranol WS | 5.000 | 4.664 | 6.271 |
| cocoyl taurate | (40% active) | | | |
| Glycol | Euperlan PK- | 3.000 | 2.799 | 9.408 |
| Distearate | 3000 | | | |
| Sodium Cocoyl | Hampocyl | 2.000 | 1.866 | 1.882 |
| Sarcosinate | C-30 (30% | | | |
| | active) | | | |
| Disodium | Chemcinate | 3.000 | 2.799 | 3.763 |
| laureth | DSLS (40% | | | |
| sulfosuccinate | active) | | | |
| | Total (Wet | 107.2 | 100.000 | |
| | Basis) | | | |
| | Total (Dry | | 57.183 | 100.000 |
| | Basis) | | | |

### Example II

### Fibrous Water Soluble Polymer

In this Example, cleansing sheets containing different types and combinations of fibrous water soluble polymers were evaluated. The cleansing sheets were prepared by the process described above. The results are set forth in Table 1 below.

Formulation IIA: 100% Methylcellulose

| | | | Wt. % | Wt. % |
|---|---|---|---|---|
| | | Amount | (Wet | (Dried |
| | | (grams) | Formula) | product) |
| Water | Water | 45.900 | 42.817 | 3.361 |
| Methylcellulose | Methocel | 10.000 | 9.328 | 31.354 |
| | A125LV | | | |
| Blend of Sulfates | Sulfochem | 0.200 | 0.187 | 0.314 |
| | ASV-K (50% | | | |
| | active) | | | |
| Hydroxypropylcellulose | Methocel K35 | 0.000 | 0.000 | 0.000 |
| | LV | | | |
| Sodium Laureth | Rhodapex | 27.000 | 25.187 | 22.012 |
| Sulfate | ES-2K (26% | | | |
| | active) | | | |
| Cocamidopropyl | Tegobetaine | 6.000 | 5.597 | 5.644 |
| betaine | L7-V (30% | | | |
| | active) | | | |
| Glycerin, USP | Emery 917 | 4.000 | 3.731 | 12.541 |
| Phenoxyethanol & | Phenonip | 0.300 | 0.280 | 0.941 |
| parabens | | | | |
| Fragrance | IFF 303 AV | 0.800 | 0.746 | 2.508 |
| | PCM RPD | | | |
| Sodium methyl cocoyl | Tauranol WS | 5.000 | 4.664 | 6.271 |
| taurate | (40% active) | | | |
| Glycol Distearate | Euperlan PK- | 3.000 | 2.799 | 9.408 |
| | 3000 | | | |
| Sodium Cocoyl | Hampocyl C- | 2.000 | 1.866 | 1.882 |
| Sarcosinate | 30 (30% | | | |
| | active) | | | |
| Disodium laureth | Chemcinate | 3.000 | 2.799 | 3.763 |
| sulfosuccinate | DSLS (40% | | | |
| | active) | | | |
| | Total (Wet | 107.200 | 100.000 | |
| | Basis) | | | |
| | Total (Dry | | 57.183 | 100.000 |
| | Basis) | | | |

Formulation IIB: 100% Hydroxypropylcellulose

| | | | Wt. % | Wt. % |
|---|---|---|---|---|
| | | Amount | (Wet | (Dried |
| | | (grams) | Formula) | product) |
| Water | Water | 45.900 | 42.817 | 3.361 |
| Methylcellulose | Methocel | 0.000 | 0.000 | 0.000 |
| | A125LV | | | |
| Blend of Sulfates | Sulfochem | 0.200 | 0.187 | 0.314 |
| | ASV-K | | | |
| | (50% active) | | | |
| Hydroxypropylcellulose | Methocel | 10.000 | 9.328 | 31.354 |
| | K35 LV | | | |
| Sodium Laureth | Rhodapex | 27.000 | 25.187 | 22.012 |
| Sulfate | ES-2K | | | |
| | (26% active) | | | |
| Cocamidopropyl | Tegobetaine | 6.000 | 5.597 | 5.644 |
| betaine | L7-V | | | |
| | (30% active) | | | |
| Glycerin, USP | Emery 917 | 4.000 | 3.731 | 12.541 |
| Phenoxyethanol & | Phenonip | 0.300 | 0.280 | 0.941 |
| parabens | | | | |
| Fragrance | IFF 303 | 0.800 | 0.746 | 2.508 |
| | AV PCM | | | |
| | RPD | | | |
| Sodium methyl cocoyl | Tauranol WS | 5.000 | 4.664 | 6.271 |
| taurate | (40% active) | | | |
| Glycol Distearate | Euperlan PK- | 3.000 | 2.799 | 9.408 |
| | 3000 | | | |
| Sodium Cocoyl | Hampocyl | 2.000 | 1.866 | 1.882 |
| Sarcosinate | C-30 (30% | | | |
| | active) | | | |
| Disodium laureth | Chemcinate | 3.000 | 2.799 | 3.763 |
| sulfosuccinate | DSLS (40% | | | |
| | active) | | | |
| | Total (Wet | 107.200 | 100.000 | |
| | Basis) | | | |
| | Total (Dry | | 57.183 | 100.000 |
| | Basis) | | | |

Formulation IIC: 4:1 Ratio of Methylcellullose to Hydroxypropylcellulose

| | | | Wt. % | Wt. % |
|---|---|---|---|---|
| | | Amount | (Wet | (Dried |
| | | (grams) | Formula) | product) |
| Water | Water | 45.900 | 42.817 | 3.361 |
| Methylcellulose | Methocel A125LV | 8.000 | 7.463 | 25.085 |
| Blend of | Sulfochem ASV-K | 0.200 | 0.187 | 0.314 |
| Sulfates | (50% active) | | | |
| Hydroxypropyl- | Methocel K35 LV | 2.000 | 1.866 | 6.272 |
| cellulose | | | | |
| Sodium Laureth | Rhodapex ES-2K | 27.000 | 25.187 | 22.011 |
| Sulfate | (26% active) | | | |
| Cocamidopropyl | Tegobetaine L7-V | 6.000 | 5.597 | 5.644 |
| betaine | (30% active) | | | |
| Glycerin, USP | Emery 917 | 4.000 | 3.731 | 12.541 |
| Phenoxyethanol | Phenonip | 0.300 | 0.280 | 0.941 |
| & parabens | | | | |
| Fragrance | IFF 303 AV | 0.800 | 0.746 | 2.507 |
| | PCM RPD | | | |
| Sodium methyl | Tauranol WS | 5.000 | 4.664 | 6.271 |
| cocoyl taurate | (40% active) | | | |
| Glycol | Euperlan PK- | 3.000 | 2.799 | 9.408 |
| Distearate | 3000 | | | |
| Sodium Cocoyl | Hampocyl C-30 | 2.000 | 1.866 | 1.882 |
| Sarcosinate | (30% active) | | | |
| Disodium | Chemcinate | 3.000 | 2.799 | 3.763 |
| laureth | DSLS (40% | | | |
| sulfosuccinate | active) | | | |
| | Total (Wet Basis) | 107.2 | 100.000 | |
| | Total (Dry Basis) | | 57.183 | 100.000 |

Formulation IID: 1:1 Ratio of Methylcellullose to Hydroxypropylcellulose

| | | | Wt. % | Wt. % |
|---|---|---|---|---|
| | | Amount | (Wet | (Dried |
| | | (grams) | Formula) | product) |
| Water | Water | 45.900 | 42.817 | 3.361 |
| Methylcellulose | Methocel A125LV | 5.000 | 4.664 | 15.677 |
| Blend of | Sulfochem ASV-K | 0.200 | 0.187 | 0.314 |
| Sulfates | (50% active) | | | |
| Hydroxypropyl- | Methocel K35 LV | 5.000 | 4.664 | 15.677 |
| cellulose | | | | |
| Sodium Laureth | Rhodapex ES-2K | 27.000 | 25.187 | 22.011 |
| Sulfate | (26% active) | | | |
| Cocamidopropyl | Tegobetaine L7-V | 6.000 | 5.597 | 5.644 |
| betaine | (30% active) | | | |
| Glycerin, USP | Emery 917 | 4.000 | 3.731 | 12.541 |
| Phenoxyethanol | Phenonip | 0.300 | 0.280 | 0.941 |
| & parabens | | | | |
| Fragrance | IFF 303 AV | 0.800 | 0.746 | 2.507 |
| | PCM RPD | | | |
| Sodium methyl | Tauranol WS | 5.000 | 4.664 | 6.271 |
| cocoyl taurate | (40% active) | | | |
| Glycol | Euperlan PK- | 3.000 | 2.799 | 9.408 |
| Distearate | 3000 | | | |
| Sodium Cocoyl | Hampocyl C-30 | 2.000 | 1.866 | 1.882 |
| Sarcosinate | (30% active) | | | |
| Disodium | Chemcinate | 3.000 | 2.799 | 3.763 |
| laureth | DSLS (40% | | | |
| sulfosuccinate | active) | | | |
| | Total (Wet Basis) | 107.2 | 100.000 | |
| | Total (Dry Basis) | | 57.183 | 100.000 |

Formulation IIE: 1:4 Ratio of Methylcellullose to Hydroxypropylcellulose

| | | | Wt. % | Wt. % |
|---|---|---|---|---|
| | | Amount | (Wet | (Dried |
| | | (grams) | Formula) | product) |
| Water | Water | 45.900 | 42.817 | 3.361 |
| Methylcellulose | Methocel | 2.000 | 1.866 | 6.272 |
| | A125LV | | | |
| Blend of | Sulfochem | 0.200 | 0.187 | 0.314 |
| Sulfates | ASV-K | | | |
| | (50% active) | | | |
| Hydroxypropylcellulose | Methocel | 8.000 | 7.463 | 25.085 |
| | K35 LV | | | |
| Sodium Laureth | Rhodapex | 27.000 | 25.187 | 22.011 |
| Sulfate | ES-2K | | | |
| | (26% active) | | | |
| Cocamidopropyl | Tegobetaine | 6.000 | 5.597 | 5.644 |
| betaine | L7-V | | | |
| | (30% active) | | | |
| Glycerin, USP | Emery 917 | 4.000 | 3.731 | 12.541 |
| Phenoxyethanol | Phenonip | 0.300 | 0.280 | 0.941 |
| & parabens | | | | |
| Fragrance | IFF 303 AV | 0.800 | 0.746 | 2.507 |
| | PCM RPD | | | |
| Sodium methyl | Tauranol WS | 5.000 | 4.664 | 6.271 |
| cocoyl taurate | (40% active) | | | |
| Glycol | Euperlan PK- | 3.000 | 2.799 | 9.408 |
| Distearate | 3000 | | | |
| Sodium Cocoyl | Hampocyl | 2.000 | 1.866 | 1.882 |
| Sarcosinate | C-30 | | | |
| | (30% active) | | | |
| Disodium | Chemcinate | 3.000 | 2.799 | 3.763 |
| laureth | DSLS (40% | | | |
| sulfosuccinate | active) | | | |
| | Total (Wet | 107.2 | 100.000 | |
| | Basis) | | | |
| | Total (Dry | | 57.183 | 100.000 |
| | Basis) | | | |

TABLE I
Comparison of Fibrous Water Soluble Polymers
The cleansing sheets prepared in Example II were compared and the results
are summarized below:

| | Fibrous water soluble | |
|---|---|---|
| Formulation | polymer | Remarks |
| II A | 100% Methylcellulose | Unacceptable for processing because the |
| | | sheet “curled” after drying. |
| II B | 100% | Unacceptable for packaging because |
| | Hydroxypropylcellulose | product “ripped” and was mottled in |
| | | appearance. Product is likely to crack after |
| | | packaging and have a high potential for |
| | | breaking when removing from the package. |
| II C | 4:1 Ratio of | Acceptable for batch preparation, |
| | Methylcellulose to | processing into sheets and appearance. |
| | Hydroxypropylcellulose | |
| II D | 1:1 Ratio of | Still acceptable for processing and |
| | Methylcellulose to | functionality but it had a wax paper like |
| | Hydroxypropylcellulose | appearance |
| II E | 1:4 Ratio of | Formula was very thick and pasty during |
| | Methylcellulose to | batch preparation. It was very difficult to |
| | Hydroxypropylcellulose | mix and apply onto the paper before drying. |
| | | Product was more “brittle” than formulation IIC. |

### Example III

### Glycerin Plasticizer

In this example, cleansing sheets based on Example I (control) were prepared using varying amounts of glycerine. The cleansing sheets were prepared by the process described above. The results are set forth in Table 2 below.

Formulation IIIA: 0.25 g Glycerine

| | | | III A | III A |
|---|---|---|---|---|
| | | | Percentage | Percentage |
| | | Amount | (Wet | (Dried |
| | | (grams) | Formula) | product) |
| Water | Water | 49.650 | 46.315 | 3.809 |
| Methylcellulose | Methocel | 8.000 | 7.463 | 28.427 |
| | A125LV | | | |
| Blend of Sulfates | Sulfochem | 0.200 | 0.187 | 0.356 |
| | ASV-K | | | |
| | (50% active) | | | |
| Hydroxypropylcellulose | Methocel | 2.000 | 1.866 | 7.108 |
| | K35 LV | | | |
| Sodium Laureth | Rhodapex | 27.000 | 25.187 | 24.944 |
| Sulfate | ES-2K | | | |
| | (26% active) | | | |
| Cocamidopropyl | Tegobetaine | 6.000 | 5.597 | 6.396 |
| betaine | L7-V | | | |
| | (30% active) | | | |
| Glycerin, USP | Emery 917 | 0.250 | 0.233 | 0.888 |
| Phenoxyethanol & | Phenonip | 0.300 | 0.280 | 1.067 |
| parabens | | | | |
| Fragrance | IFF 303 | 0.800 | 0.746 | 2.842 |
| | AV PCM | | | |
| | RPD | | | |
| Sodium methyl cocoyl | Tauranol | 5.000 | 4.664 | 7.106 |
| taurate | WS (40% | | | |
| | active) | | | |
| Glycol Distearate | Euperlan | 3.000 | 2.799 | 10.662 |
| | PK-3000 | | | |
| Sodium Cocoyl | Hampocyl | 2.000 | 1.866 | 2.132 |
| Sarcosinate | C-30 (30% | | | |
| | active) | | | |
| Disodium laureth | Chemcinate | 3.000 | 2.799 | 4.265 |
| sulfosuccinate | DSLS (40% | | | |
| | active) | | | |
| | Total (Wet | 107.200 | 100.000 | |
| | Basis) | | | |
| | Total (Dry | | | 100.000 |
| | Basis) | | | |

Formulation IIIB: 10 g Glycerine

| | | | | Wt. % |
|---|---|---|---|---|
| | | Amount | Wt. % (Wet | (Dried |
| | | (grams) | Formula) | product) |
| Water | Water | 39.900 | 37.220 | 2.829 |
| Methylcellulose | Methocel A125LV | 8.000 | 7.463 | 21.113 |
| Blend of Sulfates | Sulfochem ASV-K (50% | 0.200 | 0.187 | 0.265 |
| | active) | | | |
| Hydroxypropylcellulose | Methocel K35 LV | 2.000 | 1.866 | 5.279 |
| Sodium Laureth | Rhodapex ES-2K (26% | 27.000 | 25.187 | 18.526 |
| Sulfate | active) | | | |
| Cocamidopropyl | Tegobetaine L7-V (30% | 6.000 | 5.597 | 4.750 |
| betaine | active) | | | |
| Glycerin, USP | Emery 917 | 10.000 | 9.328 | 26.389 |
| Phenoxyethanol & | Phenonip | 0.300 | 0.280 | 0.792 |
| parabens | | | | |
| Fragrance | IFF 303 AV PCM RPD | 0.800 | 0.746 | 2.110 |
| Sodium methyl cocoyl | Tauranol WS (40% active) | 5.000 | 4.664 | 5.278 |
| taurate | | | | |
| Glycol Distearate | Euperlan PK-3000 | 3.000 | 2.799 | 7.918 |
| Sodium Cocoyl | Hampocyl C-30 (30% | 2.000 | 1.866 | 1.584 |
| Sarcosinate | active) | | | |
| Disodium laureth | Chemcinate DSLS (40% | 3.000 | 2.799 | 3.167 |
| sulfosuccinate | active) | | | |
| | Total (Wet Basis) | 107.200 | 100.000 | 100.000 |
| | Total (Dry Basis) | | | |

TABLE 2
Effect of Glycerine

| Formulation | |
|---|---|
| IIIA | Compared to the control |
| | (Example 1): |
| | Base was very “fluffy” |
| | and could not be easily |
| | applied to the paper |
| | prior to drying. |
| | Base was not pourable |
| | More difficult to release |
| | from the paper. Would |
| | be a production issue |
| IIIB | Compared to the control |
| | (Example 1): |
| | Base was more “shiny” |
| | and pourable |
| | Appearance was more |
| | “translucent” than the |
| | control |
| | More difficult to dissolve |
| | but acceptable. |
| | Very dense foam and |
| | felt “creamy” - felt that |
| | it helped moisturization |
| | Product did not curl |

### Example IV

A single use cleansing sheet containing salicylic acid was prepared by the above process and containing the ingredients set forth in the following table:

| | | | Percentage | Percentage |
|---|---|---|---|---|
| | | | (Wet | (Dried |
| | | Amount (grams) | Formula) | product) |
| Water | Water | 45.900 | 44.092 | 3.401 |
| Methylcellulose | Methocel A125LV | 8.000 | 7.685 | 26.136 |
| Blend of Sulfates | Sulfochem ASV-K | 0.100 | 0.096 | 0.163 |
| | (50% active) | | | |
| Hydroxypropylcellulose | Methocel K35 LV | 2.000 | 1.921 | 6.533 |
| Sodium Laureth | Rhodapex ES-2K | 27.000 | 25.937 | 22.934 |
| Sulfate | (26% active) | | | |
| Cocamidopropyl | Tegobetaine L7-V | 6.000 | 5.764 | 5.881 |
| betaine | (30% active) | | | |
| Glycerin, USP | Emery 917 | 4.000 | 3.842 | 13.066 |
| Phenoxyethanol & | Phenonip | 0.300 | 0.288 | 0.979 |
| parabens | | | | |
| Fragrance | IFF 303 AV PCM | 0.800 | 0.768 | 2.612 |
| | RPD | | | |
| Sodium methyl cocoyl | Tauranol WS | 5.000 | 4.803 | 6.534 |
| taurate | (40% active) | | | |
| Glycol Distearate | Euperlan PK- | 3.000 | 2.882 | 9.801 |
| | 3000 | | | |
| Sodium Cocoyl | Hampocyl C-30 | 2.000 | 1.921 | 1.960 |
| Sarcosinate | (30% active) | | | |
| | Salicylic Acid | | | |
| | Total (Wet Basis) | 104.100 | 100.000 | |
| | Total (Dry Basis) | | | 100.000 |

### Example V

A single use cleansing sheet containing polyethylene beads Indocus 14/2 was prepared by the above process and containing the ingredients set forth in the following table:

| | | | VII B | VII B |
|---|---|---|---|---|
| | | VII B | Percentage | Percentage |
| | | Amount | (Wet | (Dried |
| | | grams | Formula) | product) |
| Water | Water | 45.900 | 44.008 | 3.385 |
| Methylcellulose | Methocel A125LV | 8.000 | 7.670 | 25.962 |
| Blend of Sulfates | Sulfochem ASV-K (50% active) | 0.100 | 0.096 | 0.162 |
| Hydroxypropylcellulose | Methocel K35 LV | 2.000 | 1.918 | 6.492 |
| Sodium Laureth | Rhodapex ES-2K (26% active) | 27.000 | 25.887 | 22.780 |
| Sulfate | | | | |
| Cocamidopropyl | Tegobetaine L7-V (30% active) | 6.000 | 5.753 | 5.842 |
| betaine | | | | |
| Glycerin, USP | Emery 917 | 4.000 | 3.835 | 12.981 |
| Phenoxyethanol & | Phenonip | 0.300 | 0.288 | 0.975 |
| parabens | | | | |
| Fragrance | IFF 303 AV PCM RPD | 0.800 | 0.767 | 2.596 |
| Sodium methyl cocoyl | Tauranol WS (40% active) | 5.000 | 4.794 | 6.491 |
| taurate | | | | |
| Glycol Distearate | Euperlan PK-3000 | 3.000 | 2.876 | 9.735 |
| Sodium Cocoyl | Hampocyl C-30 (30% active) | 2.000 | 1.918 | 1.948 |
| Sarcosinate | Salicylic Acid | | 0.000 | 0.000 |
| Beads | Indocus 14/2 | 0.200 | 0.192 | 0.650 |
| | Total (Wet Basis) | 104.300 | 100.000 | |
| | Total (Dry Basis) | | | 100.000 |

### Example VI

A single use cleansing sheet containing oatmeal was prepared by the above process and containing the ingredients set forth in the following table:

| | | 1.0 gms | | |
|---|---|---|---|---|
| | | Oatmeal | VII C | VII C |
| | | VII C | Percentage | Percentage |
| | | Amount | (Wet | (Dried |
| | | grams | Formula) | product) |
| Water | Water | 45.900 | 43.673 | 3.324 |
| Methylcellulose | Methocel A125LV | 8.000 | 7.612 | 25.302 |
| Blend of Sulfates | Sulfochem ASV-K (50% active) | 0.100 | 0.095 | 0.158 |
| Hydroxypropylcellulose | Methocel K35 LV | 2.000 | 1.903 | 6.326 |
| Sodium Laureth | Rhodapex ES-2K (26% active) | 27.000 | 25.690 | 22.201 |
| Sulfate | | | | |
| Cocamidopropyl | Tegobetaine L7-V (30% active) | 6.000 | 5.709 | 5.693 |
| betaine | | | | |
| Glycerin, USP | Emery 917 | 4.000 | 3.806 | 12.651 |
| Phenoxyethanol & | Phenonip | 0.300 | 0.285 | 0.947 |
| parabens | | | | |
| Fragrance | IFF 303 AV PCM RPD | 0.800 | 0.761 | 2.530 |
| Sodium methyl cocoyl | Tauranol WS (40% active) | 5.000 | 4.757 | 6.324 |
| taurate | | | | |
| Glycol Distearate | Euperlan PK-3000 | 3.000 | 2.854 | 9.487 |
| Sodium Cocoyl | Hampocyl C-30 (30% active) | 2.000 | 1.903 | 1.898 |
| Sarcosinate | Oatmeal | 1.000 | 0.951 | 3.161 |
| | Total (Wet Basis) | 105.100 | 100.000 | |
| | Total (Dry Basis) | | | 100.000 |

### Example VII

A single use cleansing sheet containing CHEMYSOAP was prepared by the above process and containing the ingredients set forth in the following table:

| | | | VII E | VII E |
|---|---|---|---|---|
| | | VII E | Percentage | Percentage |
| | | Amount | (Wet | (Dried |
| | | grams | Formula) | product) |
| Water | Water | 45.900 | 42.461 | 3.120 |
| Methylcellulose | Methocel A125LV | 8.000 | 7.401 | 23.089 |
| Blend of Sulfates | Sulfochem ASV-K (50% active) | 0.100 | 0.093 | 0.145 |
| Hydroxypropylcellulose | Methocel K35 LV | 2.000 | 1.850 | 5.771 |
| Sodium Laureth | Rhodapex ES-2K (26% active) | 27.000 | 24.977 | 20.259 |
| Sulfate | | | | |
| Cocamidopropyl | Tegobetaine L7-V (30% active) | 6.000 | 5.550 | 5.194 |
| betaine | | | | |
| Glycerin, USP | Emery 917 | 4.000 | 3.700 | 11.543 |
| Phenoxyethanol & | Phenonip | 0.300 | 0.278 | 0.867 |
| parabens | | | | |
| Fragrance | IFF 303 AV PCM RPD | 0.800 | 0.740 | 2.309 |
| Sodium methyl cocoyl | Tauranol WS (40% active) | 5.000 | 4.625 | 5.771 |
| taurate | | | | |
| Glycol Distearate | Euperlan PK-3000 | 3.000 | 2.775 | 8.657 |
| Sodium Cocoyl | Hampocyl C-30 (30% active) | 2.000 | 1.850 | 1.731 |
| Sarcosinate | Chemysoap | 4.000 | 3.700 | 11.543 |
| | Total (Wet Basis) | 108.100 | 100.000 | |
| | Total (Dry Basis) | | | 100.000 |

### Example VIII

### Effect of Sulfochem on Viscosity

In this Example a cleansing sheet was prepared according to the process described above. The cleansing sheet had the ingredients described below.

| | | No | | |
|---|---|---|---|---|
| | | Sulfochem | | |
| | | ASV-K | V A | V A |
| | | V A | Percentage | Percentage |
| | | Amount | (Wet | (Dried |
| | | (grams) | Formula) | product) |
| Water | Water | 46.100 | 43.004 | 3.372 |
| Methylcellulose | Methocel A125LV | 8.000 | 7.463 | 25.164 |
| Blend of Sulfates | Sulfochem ASV-K (50% active) | 0.000 | 0.000 | 0.000 |
| Hydroxypropylcellulose | Methocel K35 LV | 2.000 | 1.866 | 6.292 |
| Sodium Laureth Sulfate | Rhodapex ES-2K (26% active) | 27.000 | 25.187 | 22.081 |
| Cocamidopropyl betaine | Tegobetaine L7-V (30% active) | 6.000 | 5.597 | 5.662 |
| Glycerin, USP | Emery 917 | 4.000 | 3.731 | 12.580 |
| Phenoxyethanol & | Phenonip | 0.300 | 0.280 | 0.944 |
| parabens | | | | |
| Fragrance | IFF 303 AV PCM RPD | 0.800 | 0.746 | 2.515 |
| Sodium methyl cocoyl | Tauranol WS (40% active) | 5.000 | 4.664 | 6.290 |
| taurate | | | | |
| Glycol Distearate | Euperlan PK-3000 | 3.000 | 2.799 | 9.438 |
| Sodium Cocoyl | Hampocyl C-30 (30% active) | 2.000 | 1.866 | 1.888 |
| Sarcosinate | | | | |
| Disodium laureth | Chemcinate DSLS (40% active) | 3.000 | 2.799 | 3.775 |
| sulfosuccinate | | | | |
| | Total (Wet Basis) | 107.200 | 100.000 | |
| | Total (Dry Basis) | | | 100.000 |

Prior to drying the viscosity was measured using a Brookfield Viscometer, LVT DV-1 viscometer, # 3 spindle, 6 rpm. The viscosity was 16,800 cps; pH 6.55. The same formulation with 0.2% water replaced with 0.2% Sulfochem was prepared. The viscosity was measured using the same procedure at +/−14,270 cps; pH 6.48. A third formulation was prepared replacing 0.75% water with 0.75% Sulfochem and the viscosity was measured as +/−12,560 cps; pH 6.45. Accordingly, the addition of Sulfochem decreases the viscosity.

### Example IX

### Product Solubility

In this Example, the effect of the incorporation of “air” into the batch on product solulbility was evaluated. The results are shown in Table 3 below. A formulation containing the following ingredients was prepared according to the process described above.

| Ingredients | Trade Name | Percentage |
|---|---|---|
| Water | DI Water | 39.7 |
| Methocel - E5 | E5 LV Hydroxypropyl | 10.0 |
| | methylcellulose | |
| Blends of Sulfates | Sulfochem ASV-K | 0.2 |
| Sodium Laureth Ether | Rhodapex ES-2K | 27.0 |
| Sulfate | | |
| Cocamidopropyl betaine | Tegobetaine | 6.0 |
| Glycerin, USP | Emery 917 | 4.0 |
| Phenoxyethanol & | Phenonip | 0.3 |
| Parabens | | |
| Fragrance | IFF 303 | 0.8 |
| Sodium Methyl Cocoyl | Tauranol WS | 5.0 |
| Turate | | |
| Glycol Disterate | Euperian PK-3000 | 3.0 |
| Disodium Laureth | Ultrasurf DSLS | 3.0 |
| Sulfosuccinate | | |
| Salicylic Acid | | 0.5 |
| Beads | Arch Cirebelle 104 | 0.5 |

Prior to drying, the above formula was split into five equally sized smaller batches. The “specific gravity” was measured with a Fischer Scientific Pycnometer initially and after homogenizing air into the batch. As the data shows below, the sheet solubility will improve as the specifically gravity of the feedstock is reduced.

The dissolvability was tested by putting the product in the hands, wetting the product with warm water, and rubbing the product between the hands to provide mechanical action to help product disintegration into minute particles. The dissolvability rate was rated on a 10 point scale with zero (0) being no dissolvability and 10 being “extremely fast” dissolvability.

TABLE 3
As demonstrated below, it was discovered that product solubility
can be dramatically improved by reducing the “specific gravity”
of the formula by homogenizing the batch.

| | Specific | | |
|---|---|---|---|
| | Gravity of | Dissolvability | |
| Batch No. | Basestock | Rating | Comments |
| IXA | 0.964 | 4 | Poor dissolvability |
| IXB | 0.8756 | 6 | Slight improvement over FZ-103 |
| IXC | 0.7590 | 8 | Slight improvement over FZ-104 |
| IXD | 0.7140 | 9 | Moderate initial residue but |
| | | | dissolved easily when rubbed |
| | | | between the hands |
| IXE | 0.6982 | 10 | Best dissolvability |

### Example X

### Product Solubility

In this example, batch nos. IX A, B, C and E were additionally evaluated for improved solubility using the following procedure.

Procedure
1. 500 grams of 35° C. tap water was put into a 1 liter beaker.
2. A stirrer with a 1¾″ impeller was placed into the water. The impeller blade was ¼ inch off of the bottom and ¼ inch from the side wall.
3. The mixer was turned on and the mixer speed was set at 268 RPM. The sample was put into the agitating water. The time was recorded when the sample made contact with the water.
4. The sample was mixed for 1 minute. At this point a spatula was put into the top surface of the agitating liquid, and moved back and forth, in order to separate the solids from the foam that was generated (so the solids can be easily seen).
5. When the solids were totally solubilized in the water the time was noted and recorded.

The results are set forth in the table below. Product solubility improved as the specific gravity was reduced.

| | | | Time For | | |
|---|---|---|---|---|---|
| | Water | Mixer | Total | Sample | Base stock |
| | Temp. | Speed | Solubilization | Weight | “Specific |
| Batch # | (° C.) | (RPM) | (seconds) | (grams) | Gravity” |
| (IXA) | 35 | 268 | 155 | 0.2976 | 0.964 |
| (IXB) | 35 | 269 | 105 | 0.2987 | 0.8756 |
| (IXC) | 35 | 269 | 100 | 0.2822 | 0.7590 |
| (IXE) | 35 | 268 | 95 | 0.2794 | 0.6960 |

### Example XI

### Product Pliability

In this example, the effect of the type and combination of fibrous water soluble polymer on product “pliability” was evaluated.

| Ingredients | Trade Name | Percentage | Percentage |
|---|---|---|---|
| Water | DI Water | 39.7 | 39.7 |
| Methocel - E5 | E5 LV | 10.0 | 5.0 |
| | Hydroxypropyl | | |
| | methylcellulose | | |
| Methocel K35LV | Hydroxypropyl | 0 | 5.0 |
| | cellulose | | |
| Blends of | Sulfochem ASV-K | 0.2 | 0.2 |
| Sulfates | | | |
| Sodium Laureth | Rhodapex ES- | 27.0 | 27.0 |
| Ether Sulfate | 2K | | |
| Cocamidopropyl | Tegobetaine | 6.0 | 6.0 |
| betaine | | | |
| Glycerin, USP | Emery 917 | 4.0 | 4.0 |
| Phenoxyethanol | Phenonip | 0.3 | 0.3 |
| & Parabens | | | |
| Fragrance | IFF 303 | 0.8 | 0.8 |
| Sodium Methyl | Tauranol WS | 5.0 | 5.0 |
| Cocoyl Turate | | | |
| Glycol Disterate | Euperian PK- | 3.0 | 3.0 |
| | 3000 | | |
| Disodium Laureth | Ultrasurf DSLS | 3.0 | 3.0 |
| Sulfosuccinate | | | |
| Salicylic Acid | | 0.5 | 0.5 |
| Beads | Arch Cirebelle | 0.5 | 0.5 |
| | 104 | | |

Using only hydroxypropyl methyl cellulose resulted in a product that was very “brittle”, i.e., not pliable (it will break if the product is bent when handling or removed from a package). However, when hydroxypropyl cellulose was used in combination with hydroxypropyl methylcellulose the product was significantly better in pliability and overall brittleness was reduced.

### Example XII

A single use moisturizing lotion sheet was prepared by the following process and containing the ingredients set forth in the following table:
Step 1. Added water to compounding tank and started the turbine and sweep agitation when the water level was sufficient to allow mixing.
Step 2. While the water was being added and mixing started, the fibrous water soluble polymers were added. Mixing continued for 30 minutes using high turbine agitation but avoiding aeration. After 30 minutes the batch was checked for undissolved. Mixing continued if the fibrous water soluble polymers were not completely dispersed.
Step 3. Reduced turbine agitation and began heating the batch to 80-82° C. Added Glycerin.
Step 5. When the batch temperature was between 80 and 82° C. heating stopped and the following ingredients were added:
   Cetyl Alcohol
   Brij 721
   Brij 72
   Esp ES-DF
   Sulfochem ASV-K
   Tauranol WS
Step 6. This temperature was maintained for 15 minutes or until all the solids were dissolved, whichever is greater. Checked for undissolved particulates.
Step 7 Began cooling to 40° C. at the rate of approximately 1° C. per minute. Adjusted turbine and sweep agitation as needed to facilitate cooling.

Step 8. At 40-43° C., added preservative and Fragrance. Mixed for at least 20 minutes while continuing to cool to 25-30° C.

| | | | Wt. % | Wt. % |
|---|---|---|---|---|
| | | Amount | (Wet | (Dried |
| | | grams | Formula) | product) |
| Water | Water | 65.900 | 45.401 | 3.020 |
| Methylcellulose | Methocel A125LV | 8.000 | 5.512 | 16.645 |
| Blend of Sulfates | Sulfochem ASV-K (50% active) | 0.100 | 0.069 | 0.104 |
| Hydroxypropylcellulose | Methocel K35 LV | 2.000 | 1.378 | 4.161 |
| Sodium Laureth | Rhodapex ES-2K (26% active) | 0.000 | 0.000 | 0.000 |
| Sulfate | | | | |
| Cocamidopropyl | Tegobetaine L7-V (30% active) | 60.000 | 41.337 | 54.357 |
| betaine | | | | |
| Glycerin, USP | Emery 917 | 2.000 | 1.378 | 4.161 |
| Phenoxyethanol & | Phenonip | 0.500 | 0.344 | 1.039 |
| parabens | | | | |
| Fragrance | IFF 303 AV PCM RPD | 0.800 | 0.551 | 1.664 |
| Sodium methyl cocoyl | Tauranol WS (40% active) | 0.000 | 0.000 | 0.000 |
| taurate | | | | |
| Glycol Distearate | Euperlan PK-3000 | 3.000 | 2.067 | 6.242 |
| Sodium Cocoyl | Hampocyl C-30 (30% active) | 0.000 | 0.000 | 0.000 |
| Sarcosinate | | | | |
| Stearth-2 | Brij 72 | 0.400 | 0.276 | 1.208 |
| Stearth-21 | Brij 721 | 0.250 | 0.172 | 0.755 |
| Cetyl alcohol | Crodacol C95 | 1.200 | 0.827 | 3.624 |
| Petrolatum | ESP ES-DF | 1.000 | 0.689 | 3.020 |
| | Total (Wet Basis) | 145.150 | 100.000 | |
| | Total (Dry Basis) | | | 100.000 |

What is claimed is:

1. A single use personal care sheet comprising:
(a) a fibrous water soluble polymer portion comprising (i) methylcellulose and (ii) at least one other fibrous water soluble polymer selected from hydroxypropyl cellulose, ethyl 2-hydroxyethyl ether cellulose, hydroxypropyl methyl cellulose, carboxycellulose, polyvinyl alcohol and polyacrylates, wherein the ratio of methylcellulose to said at least one other fibrous water soluble polymer is effective to provide a personal care sheet which wets out almost immediately upon contact with water;
(c) a plasticizer in an amount effective to increase the flexibility and strength of the sheet;
(d) a combination of at least one hydrophobic emulsifier and at least one hydophyllic emulsifier in an amount effective to provide a stable emulsion; and
(e) water.

2. A method for moisturizing the hands, face or body comprising wetting with water the cleansing sheet according to claim 1; and contacting hands, face or body with said wetted sheet.

3. A single use cleansing sheet comprising
a. a fibrous water soluble polymer portion comprising (i) methylcellulose and (ii) at least one other fibrous water soluble polymer selected from hydroxypropyl cellulose, ethyl 2-hydroxyethyl ether cellulose, hydroxypropyl methyl cellulose, carboxycellulose, polyvinyl alcohol and polyacrylates, wherein the ratio of methylcellulose to said at least one other fibrous water soluble polymer is effective to provide a cleansing sheet which wets out almost immediately upon contact with water;
b. a plasticizer present in an amount effective to increase the flexibility and strength of the sheet;
c. a surfactant portion present in an amount effective to provide cleansing and lathering properties; and
d. water.

4. The cleansing sheet according to claim 3, wherein the ratio of methylcellulose to said at least one other water soluble polymer ranges from about 4:1 to less than about 1:4.

5. The cleansing sheet according to claim 4, wherein said ratio ranges from about 4:1 to about 1:1.

6. The cleansing sheet according to claim 5, wherein said ratio is about 4:1.

7. The cleansing sheet according to claim 3, wherein said surfactant is present at from about 20-80% by weight on a dry basis (dried sheet), based on the total composition.

8. The cleansing sheet according to claim 3, wherein the amount of fiber forming water soluble ingredient ranges from 8% to 10%.

9. The cleansing sheet according to claim 3, which is free of natural soaps.

10. The cleansing sheet according to claim 3, wherein the ratio of methylcellulose to said at least one other water soluble polymer is effective to provide a cleansing sheet which wets out in less than one minute after contact with water.

11. The cleansing sheet according to claim 3, wherein the ratio of water to fibrous water soluble polymer portion ranges from about 95:5 to about 50:50.

12. The cleansing sheet according to claim 11, wherein said ratio ranges from about 80:20 to about 85:15.

13. The cleansing sheet according to claim 3, wherein the ratio of surfactant portion to fibrous water soluble polymer portion ranges from about 50:50 to about 90:10.

14. The cleansing sheet according to claim 13, wherein said ratio ranges from about 70:30 to about 80:20.

15. The cleansing sheet according to claim 3, wherein said surfactant portion is selected from sodium laureth sulfate, cocamidopropyl betaine, sodium methyl cocoyl taurate, sodium cocoyl sarcosinate, disodium laureth sulfosuccinate, sodium laureth sulfate, magnesium laureth sulfate, sodium laureth-8 sulfate, magnesium laureth 8 sulfate, sodium oleth sulfate, magnesium oleth sulfate, monoalkylphosphates, and combinations thereof.

16. A cleansing sheet according to claim 3, wherein said surfactant portion comprises:
a. sodium laureth sulfate present in a weight ratio of sodium laureth sulfate to fibrous water soluble polymer portion of from about 50:50 to about 90:10;
b. cocamidopropyl betaine present in a weight ratio of cocoamidopropyl betaine to fibrous water soluble polymer portion of from about 20:80 to about 80:20 (preferably 35:65 to 40:60)
c. Sodium methyl cocoyl taurate present in a weight ratio of sodium methyl cocoyl taurate to fibrous water soluble polymer portion of from about 10:90 to about 80:20;
d. Sodium cocoyl sarcosinate present in a weight ratio of sodium cocoyl sarcosinate to fibrous water soluble polymer portion of from about 10:90 to about 80:20;..
e. disodium laureth sulfosuccinate present in a weight ratio of disodium laureth sulfosuccinate to fibrous water soluble polymer portion of from about 10:90 to about 50:50.

17. A cleansing sheet according to claim 3, wherein said plasticizer is selected from glycerine, propylene glycol, butylene glycol, diethylene glycol, polyethylene glycol, polypropylene glycols, starches, sugars, dextrin, sorbitol, glucose amine, and salts of ethanolamines.

18. A cleansing sheet according to claim 3, comprising an acne treatment agent.

19. A cleansing sheet according to claim 18, wherein said acne treatment agent is salicylic acid.

20. A cleansing sheet according to claim 3, comrprising an exfoliating agent.

21. A cleansing sheet according to claim 20, wherein said exfoliating agent is polyethylene beads.

22. A method for cleansing the hands, face or body comprising wetting with water the cleansing sheet according to claim 3; and contacting hands, face or body with said wetted sheet.
